(19) United States
(12) Patent Application Publication
(10) Pub. No.: US 2006/0019848 A1
(43) Pub. Date: Jan. 26, 2006

(54) High gloss, non-feathering lip product

(76) Inventors: Dexin Luo, Fresh Meadows, NY (US); Tian Xiang Wang, Dix Hills, NY (US); Arlette Palo, New York, NY (US); David Walter Culhane, Wantagh, NY (US); Michael A. Castro, Medford, NY (US); Clara G. Mercado, Saddle River, NJ (US); Paula R. Frischberg, Smithtown, NY (US)

Correspondence Address: THE ESTEE LAUDER COS, INC, 125 PINELAWN ROAD, MELVILLE, NY, 11747, US

(21) Appl. No.: 11/178,824

(22) Filed: Jul. 11, 2005

Related U.S. Application Data
(60) Provisional application No. 60/587,209, filed on Jul. 12, 2004.

(51) Int. Cl. A61K 8/00

(52) U.S. Cl. 510/130

(57) ABSTRACT

The invention relates to a high gloss, non-feathering topical composition comprising at least one water-insoluble, fatty alcohol-soluble polysaccharide polymer and a liquid polymeric polyol ester.

The following invention claims priority under 35 USC 119e of U.S. provisional application 60/587,209 filed Jul. 12, 2004.

### FIELD OF THE INVENTION

The invention relates to cosmetic compositions. In particular, the invention relates to color cosmetics, particularly lip products.

### BACKGROUND OF THE INVENTION

Lip products come in a variety of forms, which vary depending upon the look desired by the user. The products may be highly pigmented, pearlescent, matte, or glossy/shiny. The latter type, i.e., the high gloss type of lip color, is currently particularly popular, especially among young women. The look conferred by such a product is highly glamorous and sensual, but has several drawbacks associated with it. The current technology available to produce a high shine lipstick is such that those lipstick or glosses giving the best shine typically have little staying power on the lips. In addition, these products also give rise to moderate to severe feathering, a condition in which the pigment intended to color the lips actually migrates into the fine lines around the mouth, producing a smeared, unattractive appearance. There are steps that can be taken to remedy this problem, such as the use of a lip liner with the high shine product, the use of a separate gloss over a matte lipstick to achieve the desired shine, or the use of various pearlescent fillers to produce a pearl-based, rather than oil-based, shine. However, the additional steps required may be unsatisfactory to some consumers, while the pearlescent shine may not be as appealing to some users as the oil-based shine.

An alternative solution is the use of controlled release materials. These materials, typically solids containing entrapped or adsorbed liquids, which act as the shine component, are applied to the lip. The liquid is released by shear force (i.e., by the force of application) or by the ultimate force of equilibrium conditions, giving a glossy appearance for a period of time. However, to date, these technologies have not yielded the type of super-shine product that is currently so much in demand.

Yet another possibility for producing shine is the use of film forming agents in the composition. Such materials do provide a certain level of gloss, but that gloss is proportional to the amount of film former used. Issues of comfort and aesthetics limit the amount of film forming agents that can be used in a lip composition, so this approach has found limited success in producing a high shine product.

It has also been recognized that a combination of two or more of these approaches may yield the desired high gloss lip product. However, the combination is more easily managed in concept than in practice: achieving compatibility of film-formers, controlled release agents and other shine-boosting components to achieve high shine, while avoiding syneresis and/or reducing feathering, has not been a simple task.

There is thus still a need for a high shine cosmetic composition that avoids the unattractive feathering and short wear problems, while providing a vivid gloss to the user's lips, all in a single product. The present invention provides a solution to this need.

### SUMMARY OF THE INVENTION

The invention provides cosmetic compositions comprising at least one water insoluble, fatty alcohol soluble polysaccharide polymer in combination with at least one polymeric liquid polyol ester. The compositions of the invention are particularly suitable for use in a lip product, providing a long-lasting high shine, with minimal or no feathering. The invention also provides a method of providing high shine to a keratinous surface comprising applying to the surface a composition comprising at least one water insoluble, fatty alcohol soluble carbohydrate polymer in combination with at least one polymeric liquid polyol ester.

### DETAILED DESCRIPTION OF THE INVENTION

The compositions of the invention comprise two basic components, a water insoluble, fatty alcohol soluble polysaccharide polymer blended with a polymeric liquid polyol ester. The combination of these two types of molecules results in a softly gelled system which, when applied to the skin, slowly releases the ester (the shine component), resulting in a longer lasting shine that has typically been achieved with other high shine cosmetics which have relied on shine from film formers or oils.

The polysaccharide polymer employed in the invention is one that is capable of gelling an oil phase, particularly a polar oil phase. The ideal polymer is one which is substantially water insoluble, but is fatty alcohol soluble. Examples of useful categories of polysaccharides are water insoluble, fatty alcohol soluble starches, glycogens, dextrins and celluloses. Particularly useful are water insoluble, fatty alcohol soluble celluloses, in particular C1-C8 alkyl-modified celluloses. In a preferred embodiment, the polymer is an ethyl cellulose. Ethyl celluloses are widely used in both cosmetic and pharmaceutical formulations, as film formers, binders, fillers, and delayed release systems. Any water-insoluble ethyl cellulose, or combinations thereof, is appropriate for use in the present invention. Particularly preferred, however, are ethyl celluloses routinely used for microencapsulation purposes. Examples of such ethylcelluloses are those sold by Dow Chemical Company under the brand name EthoceL®, Standard 45 Premium, which has a number average molecular weight (Mn) of about 15,000 and a weight average molecular weight (Mw) of about 160,000, or Ethocel®, Standard 100 Premium, with an Mn of about 20,000 and Mw of about 220,000. Other equivalent ethylcelluloses are also commercially available, such as Aqualon and Natrosol grades cellulose (Aqualon Company) and Stabilizer (TIC Gums). A preferred embodiment is one in which Ethocel® Standard 45 is combined with Ethocel® Standard 100, in a ratio of about 1.5:1. The quantities of polysaccharide polymer to be used will depend upon the desired viscosity of the final product, and thus will be a matter of choice for the user. However, as a guideline, the amount will ordinarily be from about 0.1 to about 20.0% by weight of the total composition, preferably about 0.5 to about 10.0%, and more preferably about 1 to about 5%.

The polysaccharide polymer is used in combination with at least one liquid polymeric polyol ester base, which serves as the primary shine component of the composition. The esters of interest are relatively polar solvents/emollients that are known in the art, as described, for example, in GB 2,134,538, JP 5-120209, and U.S. Pat. No. 3,694,382, the contents of which are incorporated herein by reference. The esters of interest are the products of esterification of a polyol, a monocarboxylic acid and a dicarboxylic acid, such as described in the above-referenced patent documents and WO 93/25628, also incorporated herein by reference. The polyol component of these esters is a C2-20 polyol, such as sugars or sugar alcohols, for example xylose, xylitol, erythritol, sucrose, glucose, maltose, lactose, sorbose, sorbitol, lactitol or maltitol, and derivatives thereof. Particularly useful polyols include, but are not limited to, glycerol, pentaerythritol, dipentaerythritol, tripentaerythritol, trimethylol propane, neopentyl glycol or combinations thereof.

The monocarboxylic acid component is a C4-30 carboxylic acid, which may be branched, straight chain or aromatic, or any mixture thereof. Preferably, the monocarboxylic acid is a C6-C22 monocarboxylic acid, or combinations thereof. Examples of such monocarboxylic acids are stearic, caprylic, capric, myristic, lauric, linoleic, oleic, linolenic, arachidic, arachidonic, erucic, ricinoleic, palmitic, palmitoleic and behenic acids. Particularly preferred are C6-C12 monocarboxylic acids, such as caprylic or capric, or combinations thereof.

The dicarboxylic acid component of the polyol ester may be any C2-36 straight chain, branched chain, cyclic dicarboxylic acids, or dimers of monocarboxylic fatty acids (e.g., dilinoleic acid) or combinations thereof. Preferably, a C4-C10 dicarboxylic acid, or combination thereof, is used. More preferably, the dicarboxylic acid is selected from adipic acid, succinic acid, or heptandioic acid.

As noted above, these liquid polymeric polyols, and methods of making same, are well known in the art. They are also widely commercially available. Such products can be purchased under the commercial names of Puresyn (e.g., Puresyn ME450, with a viscosity of 220 cPs at 25° C., and ME100, with a viscosity of about 1050 cPS at 25° C. from Exxon-Mobil Chemical). A particularly preferred polymeric polyol is a pentaerythrityl adipate/caprate/caprylate/heptanoate purchased from Inolex, under the trade name Lexfeel EL500. The amount of polymeric polyol employed in the composition is from about 5 to about 90% by weight of the composition, preferably from about 10 to about 75%, and more preferably about 30 to about 60%.

Although the polysaccharide polymer and the liquid polymeric polyol can be used alone in the composition, it is more often preferred to provide additional components to optimize function or for aesthetic reasons. One particularly preferred additional component is one or more fatty alcohols. The fatty alcohol increases the compatibility of the polysaccharide with the polymeric polyol, enhancing its solubility therein, and also provides additional emolliency to the product. The fatty alcohols used in the composition can be any C4-C36 fatty alcohol, preferably a C4-C22 fatty alcohol, and more preferably a C12-C22 alcohol, or any combination thereof. A particularly preferred fatty alcohol is octyldodecanol, used either alone, or in combination with another fatty alcohol. For example, octyldodecanol can be combined with a dimer fatty alcohol to increase its viscosity. A preferred dimer alcohol is a C18 dimer alcohol. The fatty alcohols are employed in an amount of from about 2 to about 80%, preferably about 5 to about 60%, and more preferably about 10 to about 30%, by weight of the composition.

Although fatty alcohols are incorporated to enhance compatibility of the polysaccharide with the polymeric polyol, it is not necessary, nor even desirable, to have complete compatibility/solubility of these two components. The objective is to achieve sufficient compatibility such that the product holds together as a single phase at least until the time of application, but not so much as to prevent their separation under the high shear conditions of application to a keratinous surface such as skin. While not wishing to be bound by any theory, it is believed that when mixed, the polysaccharide polymer may “microencapsulate” the polyol ester, providing a single homogeneous phase as a final product, but when applied to, for example, the lips, the encapsulation is disrupted by the shear force of the application process, gradually releasing the polymeric polyol ester, and thus providing a longer lasting shine. An added benefit of the composition, in the use of a polymeric polyol ester, is that, by virtue of its polarity, it has a relatively high surface tension, causing it to bead up on a lower-polarity or non-polar substrate, which has lower surface tension. Thus, the relatively polar esters, when released onto the lip, will not have as great a tendency to migrate, and will not spread much, if at all, beyond the edge of the applied film, thereby reducing feathering substantially. The use of the fatty alcohol, also relatively polar in comparison with other emollients, also reduces the capillary effect that is a major contributor to bleeding or feathering.

Although fatty alcohols are the preferred means for increasing the polysaccharide's compatibility with the ester, alternate components can also be used, either alone, or as a supplement to the fatty alcohol. If additional emolliency is desired, it may be advantageous to incorporate other polar emollients, such as liquid fatty acids (e.g., isostearic acid), or relatively polar liquid non-polymeric fatty acid esters, typically up to C18, for example neopentyl glycol diheptanoate, alkyl isononanoate, alkyl palmitate, or myristyl octanoate; esters having a free hydroxy group, such as ethyl hexyl hydroxy stearate, octyl hydroxystearate, or glyceryl mono and/or dialkyl esters; or polar vegetable oils and/or triglycerides, such as caprylic/capric triglyceride; and any mixture of the relatively polar components. Such materials not only add to the emolliency of the product, but as liquids, can also aid in improving the solubility of the polysaccharide in the polymeric ester. The amounts of these materials, if used, can be in the same range as the fatty alcohols, as described above. However, particularly with regard to fatty acids, it is possible to achieve the desired effect with relatively small amounts, e.g., in about a 1:1 ratio with the polysaccharide. Larger quantities can be employed, but with fatty acids, it is preferable to keep the amounts low due to possible irritation, but with higher amounts, this can also be countered by adding to the product an antiirritant component.

Other optional components may be incorporated, depending on the intended end use and/or form of the product. As noted above, one of the preferred and advantageous uses of the compositions is as a lip product. In this regard, the product as described above can provide a soft gel which is fully functional as a high shine lip product. However, in order to obtain a more solid product, for example, to achieve a soft stick consistency, it may be desired to add to the composition one or more thickeners and/or structuring agents. Because retaining the overall relative polarity of the composition is important to the desired performance, it is preferred not to utilize large quantities of the non-polar compounds that are traditionally utilized for structuring or thickening stick products. However, small amounts, preferably no more than 25% by weight, of such materials, for example hydrocarbon waxes, petrolatum, silicone-based resins or elastomers, or hydrocarbon polymers, such as linear or branched polybutene, polyisobutene, polyethylene, polydecene, hydrogenated derivatives thereof, and copolymers thereof, ethoxylated alcohols, or bentones, and any mixtures of the foregoing, can be employed to enhance the structure, viscosity and/or feel of the final product.

In a particularly preferred embodiment, however, a gelling system is used that is particularly adapted for thickening of polar oils. An example of such a system is the combination of at least one silica and at least one sugar fatty acid ester or ether, such as described in copending U.S. Ser. No. 60/519,583, the contents of which are incorporated herein by reference. Any silica particle can be used in the combination, provided the particle is not fully surface-coated. Use of partially coated products, while possible, will result in the need to use higher levels of silica to achieve the desired effect. If used in the composition, the amount can be up to about 40% by weight of the composition, but more typically the amount will be from about 0.1 to about 30%, preferably about 0.5 to about 10%, more preferably about 1 to about 5%, by weight of the Any type of silica, or any combination of types of silica, can be used, but the silica is preferably not completely surface coated. A particularly useful silica is a fumed silica commercially available from Cabot Corporation under the trade name Cab-O-Sil M-5.

The sugar fatty acid ester employed in the invention is a compound obtained by reacting a saturated or unsaturated C12-C22 fatty acid, preferably C16-C20 carbon atoms with a sugar or alkylsugar in which the alkyl group contains from 1 to 8 carbon atoms. The sugar is preferably a mono- or oligosaccharide. Examples of useful mono- or oligosaccharides include, but are not limited to, glucose, sucrose, galactose, fructose, lactose, mannose, maltose, trehalose, melibiose, raffinose, or ribose. A preferred sugar fatty ester is a fatty ester of glucose or alkylglucose. The fatty acid esters of alkylglucose are ethers of glucose in which the alkyl chain comprises from 1 to 8 carbon, atoms, preferably 1-4 carbon atoms. The preferred ester may contain a mixture of mono-, di-, tri- and tetraester derivatives with a proportion which may be of at least 50% by weight of mono- and diester derivatives and usually not exceeding 95% by weight of monoester derivatives relative to the total weight of the mixture. Examples of sugar fatty esters that may be used in the invention include, but are not limited to, sucrose monolaurate, glucose palmitate, alkylglucose sesquistearates, for instance methylglucose sesquistearate and alkylglucose palmitates, for instance methylglucose palmitate or ethylglucose palmitate, as well as the PEG or PPG derivatives of such compounds, for example, PEG-20 methyl glucose sesquistearate. Such compounds are widely available commercially, e.g., under the tradenames Glucate™, Glucam™, and Glucamate™ (Amerchol), Grillocose™ (Grillo-Werke), and Antil™(Goldschmidt). The amount of ester used in the composition will be from about 0.1 to about 10%, preferably about 0.5 to about 5%, by weight of the composition.

The viscosity of the end product is dependent upon the ratio of the amount of silica to the amount of sugar fatty acid ester. Overall the ratio of these materials will range from about 10:0.5 to 0.5:5.0 silica to ester, with the lower amounts of the silica producing a lower viscosity product, and higher amounts of the silica producing a higher viscosity product. A product having a ratio of about 4.0:1.0-1:1, preferably about 2:1-1:1, silica: ester is particularly preferred. The viscosity is also ultimately affected by the amount of gellant used relative to the amount of polar oil, with a higher viscosity achieved by a higher amount of gelling components. The viscosity is also affected by the polarity of the oils used, as the silica is more readily suspended in a more polar oil, presumably due to the interaction of the hydroxyl groups of the silica and the polar groups on the oils, so that a well-suspended silica can be used at smaller amounts than a silica that is not so readily suspended.

In one optional embodiment, it is possible to increase even further the lasting shine produced by the foregoing by adding to the composition one or more beads or resin particles having a coarse, rough or porous surface, and/or hollow internal cavity, which can hold any of the liquids until the high shear of application causes the liquid to be released. A particularly useful particle for this purpose is Silica MSS500/3H (Kobo). Although not in any way essential to achieve the desired high shine, such particles do appear to prolong the effect, presumably by their delayed release of emollients that contribute to the shine. Amounts, if used, are not critical, and will ordinarily be used in an amount of about 0.05 to about 5%.

Another optional ingredient for contributing to the prolonged appearance of the desired shine is a low-melting point resin or butter. By low-melting point is meant a material that melts at about normal human body temperature, so that upon application to the skin, the material gradually melts, releasing, in its liquid form, yet another source of shine. One example of such a material is stearoxy methicone/dimethicone copolymer, sold under the commercial name of Gransil ST-9 (Grant Industries).

If the product is to be used as a color cosmetic, it will also contain one or more pigments or colorants. Any type of pigment, provided it is acceptable for use in the area to which the product will be applied, and with or without surface treatment, can be used in the product of the invention: examples of useful pigments include iron oxides (yellow, red, brown or black), titanium dioxide (white), zinc oxide, chrome oxide (green), chrome hydrate (green), ultramarines, manganese violet, ferric ferrocyanide, carmine 40, ferric ammonium ferrocyanide, or combinations thereof. Interference pigments, which are thin platelike layered particles having a high refractive index, which, at a certain thickness, produce interference colors, resulting from the interference of typically two, but occasionally more, light reflections, from different layers of the plate, can also be added to provide a pearlescence to the product, is such is desired. The composition may also contain one or more types of cosmetically acceptable glitter, i.e., particles of transparent or colored, solid organic materials, such as poly (ethylene terephthalate), polymethacrylate, and poly (vinylbutyral), particles of metal, or particles of metal coated film or paper.

Organic pigments may also optionally be included; these include natural colorants and synthetic monomeric and polymeric colorants. Exemplary are phthalocyanine blue and green pigment, diarylide yellow and orange pigments, and azo-type red and yellow pigments such as toluidine red, litho red, naphthol red and brown pigments. Also useful are lakes, which are pigments formed by the precipitation and absorption of organic dyes on an insoluble base, such as alumina, barium, or calcium hydrates. Particularly preferred lakes are primary FD&C or D&C Lakes and blends thereof. Stains, such as bromo dyes and fluorescein dyes can also be employed. Pigments when used are typically present in an amount of about 0.1 to about 30%, preferably about 0.1 to about 20%, by weight of the composition.

The compositions can also contain inorganic powders, such as soft focus powders, or plate-like non-spherical powders such as bismuth oxychloride, boron nitride, barium sulfate, mica, sericite, muscovite, synthetic mica, titanium oxide coated mica, titanium oxide-coated bismuth oxychloride, titanium oxide coated talc, platelet iron oxides, metal powders such as aluminum, lauroyl lysine and platelet talc. Amounts are not critical, but if used, typically will be used in an amount of about 0.5 to about 5% The composition may also contain oil soluble active agents and skin conditioning agents. Non-limiting examples of these materials include antioxidants, ceramides, fatty acids, sunscreens, oil soluble vitamins and plant extracts, and the like. Although the most preferred embodiment of the invention is an anhydrous composition, it is possible to utilize the compositions of the invention as the oil-phase of a water and oil emulsion. When used as the oil phase of an emulsion, the composition's water phase can also contain water soluble actives.

The compositions of the invention can be used in any situation in which it is desirable to confer shine to a keratinous surface. A particularly preferred use is as a lip product, such as a lipstick, gloss, gel, or balm. However, it may also be used, either as any other type of cosmetic, with or without color, where a shine is wanted, for example, a foundation, blush, eyeshadow, eyeliner, mascara, body paint, body makeup or bronzer, or as in a skin care product, such as a sunscreen, moisturizer, or self-tanner, for application to the face or body. It can also be used in hair care or styling products, so as to add a shiny appearance to hair. In addition to the shine provided on the surfaces to which they are applied, these compositions also provides a moisturizing effect, particularly when applied to the lips.

The invention is illustrated by the following non-limiting examples.

### EXAMPLE 1

This illustrates a formula of the present invention

| | | Weight |
|---|---|---|
| Sequence | Material | percent |
| 1 | Ethylcellulose | 0.624 |
| | (Dow Chemical Ethocel Standard 100 | |
| | Premium) | |
| | Ethylcellulose | 0.936 |
| | (Dow Chemical Ethocel Standard 45 | |
| | Premium) | |
| | Pentaerythrityl adipate/ | 4.94 |
| | caprate/caprylate/heptanoate | |
| | (Lexfeel EL500) | |
| | Octyldodecanol | 2.60 |
| | Hydrogenated dilinoleyl alcohol | 3.90 |
| 2 | Silica (MSS-500/3H) | 3.00 |
| | Octyldodecanol | 12.00 |
| 3 | Silica (Cab-o-Sil M5) | 0.50 |
| | Octyldodecanol | 4.50 |
| 4 | Polyethylene | 5.25 |
| | (Performalene 400) | |
| | C20-40 Pareth-10 | 0.50 |
| | Microcrystalline wax | 2.50 |
| | Stearoxymethicone/dimethicone | 5.00 |
| | crosspolymer | |
| | Lauryl PCA | 0.50 |
| | Polybutene | 2.50 |
| | Hydrogenated dilinoleyl alcohol | 2.00 |
| | Propylparaben | 0.10 |
| | BHT | 0.05 |
| | Tocopheryl acetate | 0.10 |
| | Bis-diglyceryl polyacyladipate-2 | 7.49 |
| 5 | Ceramide 3 | 0.05 |
| | Oleic acid | 0.02 |
| | Cholesterol | 0.03 |
| 6 | Penterythrityl/adipate/ | 27.792 |
| | Caprate/caprylate/heptanoate | |
| | Cetyl PEG/PPG-10/1 dimethicone// | 0.164 |
| | Polyglyceryl-4 isostearate/hexyl | |
| | laurate | |
| | Pigment | 4.934 |
| 7 | Pigment | 7.23 |
| 8 | Methyl glucose sesquistearate | 0.790 |

To make a one kilogram batch, the ethylcellulose polymers (Sequence 1) are added into the premixed emollients and other liquids, and mixed well at room temperature. The system temperature is then raised to about 90-95° C. with 100-400 rpm mixing speed until a clear viscous soft gel is formed.

In two separate vessels, the two silicas (Sequences 2 and 3) are combined with the octyldocecanol and homogenized until smooth.

Sequences 1, 2 and 3, and all other sequences except pigments and powders, are combined in a 2 liter beaker, mixed at 92-98° C., with a speed of about 100-250 rpm until all materials are dissolved.

Sequence 6 is added to the system, which continues to be mixed until smooth.

Sequence 7 is then added and mixed until all are mixed in.

Sequence 8 is then added, and the system kept under the same conditions for at least 30 minutes, then cooled down to about 85-90° C. for pouring.

What we claim is:

1. A topical composition comprising at least one water-insoluble, fatty alcohol soluble polysaccharide polymer and a liquid polymeric polyol ester.

2. The composition of claim 1 in which the polysaccharide polymer is selected from the group consisting of starches, glycogens, dextrins and celluloses.

3. The composition of claim 1 in which the polysaccharide polymer is a C1-C8 alkyl-modified cellulose.

4. The composition of claim 1 in which the polysaccharide polymer is an ethyl cellulose.

5. The composition of claim 1 in which the ester is a reaction product of the esterification of a polyol, a monocarboxylic acid and a dicarboxylic acid.

6. The composition of claim 5 in which the polyol is a C2-C20 polyol.

7. The composition of claim 5 in which the monocarboxylic acid is a C4-C30 carboxylic acid.

8. The composition of claim 5 in which the dicarboxylic acid is a C2-C36 straight chain, branched chain or cyclic dicarboxylic acid, or a dimer of a monocarboxylic fatty acid ester.

9. The composition of claim 1 which also comprises a fatty alcohol.

10. The composition of claim 1 which also comprises a polar fatty acid or a polar fatty acid ester.

11. The composition of claim 1 which comprises a gelling system comprising at least one sugar fatty acid ester or ether and at least one silica.

12. The composition of claim 11 in which the silica is a fumed silica.

13. The composition of claim 1 which also comprises a pigment or colorant.

14. A topical composition comprising at least one water-insoluble, fatty alcohol soluble C1-C8 alkyl-modified cellulose and a polyol ester which is a reaction product of the esterification of a C2-C20 polyol, a C4-C30 monocarboxylic acid and a dicarboxylic acid selected from the group consisting of C2-C36 straight chain, branched chain or cyclic dicarboxylic acid, or a dimer of a monocarboxylic fatty acid ester.

15. The composition of claim 14 in which the polyol is selected from the group consisting of glycerol, pentaerythritol, dipentaerythritol, tripeaerythritol, trimethylol propane, neopentyl glycol, and combinations thereof, the dicarboxylic acid is selected from the group consisting of adipic acid, succinic acid and heptandioic acid, and the monocarboxylic acid is selected from stearic acid, caprylic acid, myristic acid, lauric acid, linoleic acid, linolenic acid, arachidic acid, arachidonic acid, erucic acid, ricinoleic acid, palmitic acid, palmitoleic acid, behenic acid, and combinations thereof.

16. The composition of claim 14 in which the polymeric polyol is pentaerythrityl adipate/caprate/caprylate/heptanoate.

17. The composition of claim 14 which also comprises a C12-C22 fatty alcohol.

18. The composition of claim 14 which also comprises a liquid fatty acid or a polar liquid non-polymeric fatty acid esters.

19. The composition of claim 14 which is gelled by a combination of a fumed silica and at least one sugar fatty acid ester or ether.

20. The composition of claim 14 which also comprises a pigment or colorant.
